(19) United States
(12) Patent Application Publication
(10) Pub. No.: US 2026/0360500 A1
(43) Pub. Date: Nov. 12, 2026

(54) Artificial Intelligence Hospital Decision Ventilation Settings

(71) Applicant: Brendan Ruby, Boston, MA (US)

(72) Inventor: Brendan Ruby, Boston, MA (US)

(21) Appl. No.: 19/201,896

(22) Filed: May 7, 2025

(51) Int. Cl. G16H 20/17; G16H 10/60; G16H 40/63; G16H 50/30

(52) U.S. Cl. CPC G16H 20/17; G16H 10/60; G16H 40/63; G16H 50/30

(57) ABSTRACT

This invention discloses a real-time monitoring system that detects and mitigates opioid-induced respiratory depression in clinical settings. The system integrates hospital sensors (e.g., capnography, pulse oximetry) with opioid pharmacokinetic data to assess ventilation risk dynamically. In one embodiment, a predictive algorithm calculates a risk score based on respiratory deviations and drug concentration, triggering tiered alerts for early intervention. Alternatively, an automated mitigation protocol pauses opioid infusion, suggests corrective actions, and documents events in electronic health records. The invention improves patient safety by proactively identifying ventilation conflicts, reducing reliance on threshold-based alarms.

Patients present to the emergency department (ED) with a wide range of conditions that may require tracheal intubation or positive-pressure ventilation, including pneumonia, asthma, chronic obstructive pulmonary disease (COPD), cardiogenic pulmonary edema, acute respiratory distress syndrome (ARDS), stroke, trauma, drug overdose, sepsis, shock, and neuromuscular disorders such as myasthenia gravis or Guillain-Barré syndrome.

Once a definitive airway has been secured, ventilatory management ensues. Ventilatory strategies vary according to the clinical scenario, and to provide optimal care, doctors must understand the fundamental concepts of mechanical ventilation.

Clinicians place patients on mechanical ventilation to accomplish any of the following goals, including:
To secure the airway
To improve pulmonary gas exchange (ie, reverse hypoxemia or acute respiratory acidosis)
To relieve respiratory distress (ie, decrease oxygen consumption or address respiratory muscle fatigue)
To assist with airway and lung healing
To permit appropriate sedation and neuromuscular blockade

The ventilator simulates four stages of breathing:
1. The ventilator or the patient triggers the initiation of inspiration.
2. The ventilator provides a breath, determined by preset variables (ie, pressure, volume, and flow rate).
3. The ventilator halts inspiration when a preset parameter, such as tidal volume, inspiratory time, or airway pressure, is reached.
4. The ventilator switches to expiration, and the breath is completed. Expiration occurs when the expiratory valve opens and generally involves a passive mechanism generated by the recoil of the chest wall, lungs, and diaphragm.

To manage patients requiring mechanical ventilation, emergency clinicians must be familiar with the ventilator apparatus in use at their hospital and understand the various settings that can be modified. This is how AI could be useful to this process.

Breath types—Mechanical ventilators can deliver different kinds of breaths. A mandatory breath is started, controlled, and ended by the ventilator, which does all the work. An assisted breath is initiated by the patient but controlled and ended by the ventilator. A spontaneous breath is initiated, controlled, and ended by the patient; the volume of the breath delivered by the ventilator is determined by the patient's effort, pulmonary compliance, and physiologic reserve.

Triggers—An assisted breath can be triggered by either pressure or flow. The pressure trigger requires a demand valve, which senses a negative airway pressure deflection when the patient initiates a breath. A trigger sensitivity threshold of −1 to −3 cmH₂O is common. When the threshold is reached, the ventilator delivers a breath.

The flow trigger uses a circuit of gas that flows continuously past the patient. A preset change in flow rate, created when the patient initiates a breath, triggers the ventilator to deliver a breath. A basal flow rate of 10 L/minute is common, and a trigger sensitivity threshold of 2 L/minute is frequently used.

Assist-control ventilation (ACV)—With ACV, every breath is fully supported by the ventilator, regardless of whether the breath is initiated by the patient or the machine. The clinician determines a base ventilatory rate, but the patient is able to breathe faster than this preset rate. ACV provides the patient with increased ventilatory support and reduces the work of breathing. Potential dangers include diminished cardiac output and inappropriate hyperventilation.

Synchronized intermittent mandatory ventilation (SIMV)—In SIMV mode, the patient is allowed intermittent spontaneous breaths between the mandated, preset number of ventilator-supported breaths. Spontaneous breaths above the preset ventilatory rate are not supported by the ventilator. Either the patient or the ventilator can trigger the ventilator-supported breaths.

Mechanical ventilation is associated with a number of pathophysiologic derangements that can lead to injury. Among these are:
Diminished cardiac output and hypotension
Pulmonary barotrauma (eg, pneumothorax)
Ventilator-associated lung injury (VALI)
Auto-positive end-expiratory pressure (auto-PEEP; ie, intrinsic PEEP)
Elevated intracranial pressure

Diseases and patient condition vary over time, so clinicians should regularly reassess the settings and mode of ventilation.

Trauma—Clinicians should exercise caution when initiating mechanical ventilation in the trauma patient and should consider the implications of potential hypovolemia, poor lung compliance, abdominal distention, and increased intracranial pressure (ICP).

As the need for critical care services and emergency department (ED) lengths of stay increase, emergency clinicians will increasingly be called upon to provide long-term critical care management, including weaning and discontinuation of mechanical ventilation

Objectives—Mechanical ventilation is performed in the emergency department (ED) to accomplish any of a number of goals, including the following:
To secure the airway
To improve pulmonary gas exchange (ie, reverse hypoxemia or acute respiratory acidosis)
To relieve respiratory distress (ie, decrease oxygen consumption or respiratory muscle fatigue)
To permit appropriate sedation and neuromuscular blockade

Ventilator settings—Ventilator settings are adjusted to provide adequate minute ventilation (MV) while minimizing the risk of iatrogenic complications.

Complications—Complications of mechanical ventilation include diminished cardiac output, hypotension, pulmonary barotrauma (eg, pneumothorax), ventilator-associated lung injury (VALI), ventilator-associated pneumonia (VAP), auto-positive end-expiratory pressure (auto-PEEP; ie, intrinsic PEEP), and elevated intracranial pressure (ICP).

There is no single optimal mode of mechanical ventilation; diseases and patient condition vary over time, and ventilator settings must be adjusted accordingly.

Ventilatory strategies vary according to the clinical condition or scenario; to provide optimal care, clinicians must understand fundamental concepts of mechanical ventilation. Ventilator settings can be modified, there are different modes of mechanical ventilation

Useful guidelines exist on mechanical ventilation, however clinicians must individualize mechanical ventilation based on the clinical scenario, showing how a decisional tool could be of value.

Objectives of mechanical ventilation include; to secure an airway, to improve pulmonary gas exchange, to relieve respiratory distress, to assist with airway and lung healing, to permit appropriate sedation or neuromuscular blockade.

The ventilator simulates four stages of breathing:
1. The ventilator or the patient triggers the initiation of inspiration.
2. The ventilator provides a breath, determined by preset variables (ie, pressure, volume, and flow rate),
3. The ventilator halts inspiration when a preset parameter, such as tidal volume, inspiratory time, or airway pressure, is reached.
4. The ventilator switches to expiration, and the breath is completed. Expiration occurs when the expiratory valve opens and generally involves a passive mechanism generated by the recoil of the chest wall, lungs, and diaphragm.

To manage patients requiring ventilation, clinicians must be familiar with the ventilator apparatus in use at their hospital to understand various settings that can be modified,

Ventilator settings are often modified to provide ventilation to meet metabolic demand while minimizing risk of iatrogenic complications.

Mechanical ventilators can deliver different kinds of breaths; a mandatory breath is started, controlled and ended by the ventilator, doing all the work. An assisted breath is initiated by the patient but controlled and ended by the ventilator. A spontaneous breath is initiated, controlled and ended by the patient, the volume of breath delivered by the ventilator is determined by the patients effort, pulmonary compliance, and physiologic reserve.

Triggers—An assisted breath can be triggered by either pressure or flow. The pressure trigger requires a demand valve, which senses a negative airway pressure deflection when the patient initiates a breath

The flow trigger uses a circuit of gas that flows continuously past the patient. A preset change in flow rate, created when the patient initiates a breath, triggers the ventilator to deliver a breath.

Regardless of the trigger mechanism, the threshold must be appropriate. If it is too low, the ventilator will “auto-cycle” (ie, deliver a continual series of breaths), resulting in respiratory alkalosis; if it is too high, the patient will be “locked out” from ventilator-supported breaths while expending considerable energy. In both conditions, the mechanically ventilated patient will appear to be in respiratory distress, and the work of breathing will increase.

Respiratory rate—The clinician determines the minimum number of breaths per minute (ie, respiratory rate) given to the patient by the ventilator. The rate can be adjusted according to the disease process.

Doctor determines the minimum number of breaths per minute given to the patient by the ventilator. The rate can be adjusted according to the disease process.

Modes of ventilation include ACV Assist-control ventilation, SIMV Synchronized intermittent mandatory ventilation, PSV Pressure support ventilation, NIV Positive-pressure noninvasive ventilation. ACV is used most frequently in the emergency department.

Cycling—Ventilator “cycling” refers to the mechanism by which a breath changes from inspiration to expiration. Common methods for cycling a ventilator are volume, flow, and time. Volume-cycled breaths transition from inspiration to expiration after a preset volume of gas is delivered. This is the most common form. Flow-cycled inspiratory breaths transition after a preset airway pressure is reached and flow decreases to a particular rate. Time-cycled inspiratory breaths transition after a predetermined inspiratory time. Inspiratory hold can also be included in the cycling mechanism. This function keeps air in the lungs at end-inspiration for longer intervals to maximize gas exchange.

Mechanical ventilation is associated with a number of pathophysiologic derangements that can lead to injury. Among these are:
Diminished cardiac output and hypotension
Pulmonary barotrauma (eg, pneumothorax)
Ventilator-associated lung injury (VALI)
Auto-positive end-expiratory pressure (auto-PEEP; ie, intrinsic PEEP)
Elevated intracranial pressure

clinicians can select among three fundamental strategies for mechanical ventilation: positive-pressure noninvasive ventilation (NIV) and two strategies of invasive positive-pressure ventilation (IPPV), which are low TV ventilation (LTVV; also known as lung-protective invasive positive-pressure ventilation or L-IPPV) and general IPPV.

For patients with severe asthma or COPD requiring mechanical ventilation, we recommend the following approach:
Use larger-sized endotracheal tube (ETT; eg, ≥8 mm) (patient weight or attributes suggesting difficult intubation may preclude the use of large tubes)
Keep minute ventilation (MV) below 115 ml/kg
Keep tidal volume (TV) below 8 ml/kg
Maintain respiratory rate at 10 to 14 breaths per minute
Maintain a high inspiratory flow rate of 80 to 100 L/minute
Use a lower respiratory rate and higher inspiratory flow rate to allow increased expiratory time with decreased inspiratory-to-expiratory (I:E) ratio (1:3 or 1:4 up to 1:5)
Maintain plateau pressures below 30 cm H₂O if possible
Allow hypercapnia for patients with high peak pressures

Continue comprehensive treatment of the reactive airway disease

Trauma—Clinicians should exercise caution when initiating mechanical ventilation in the trauma patient and should consider the implications of potential hypovolemia, poor lung compliance, abdominal distention, and increased intracranial pressure (ICP).

Involves an electronic computer consisting of a keyboard, mouse, electronic cord to connect to an outlet for power supply, a graphical user interface, natural language processing, artificial intelligence computer chip or processor.

Step by step process—Doctor accesses computer artificial intelligence tool holding a dilemma on patient. Next doctor inputs patient information or asks artificial intelligence tool to retrieve patient data

Patient monitoring sensors collect patient data such as heart rate, blood pressure, oxygenation levels, more. Some ways to monitor a patient include wearable devices such as a pulse oximeter, a ring that collects data on patient blood pressure, a smartwatch that collects patient blood data or oxygen data, or other monitoring sensors. Collecting or monitoring patient data as outlined, assists in decisional processes. Decisional processes sometimes require knowledge of patient blood levels or oxygen levels or CT scan data.

Patient monitoring sensors such as heart rate monitors, blood pressure monitors, pulse oximeters, to collect real-time patient data. Patient heart rate monitors continuously monitor a hospital patient to measure heart rate to detect any changes or irregularities. Patient heart rate monitors also detect abnormalities such as an irregular heartbeat that may indicate underlying conditions. Tachycardia and Bradycardia are two phrases for excessively fast or slow heart rates. Overall this device measures cardiac health through heart measurement, and alerts healthcare providers in hospitals. Portable heart rate monitors allow for mobility. Data can be integrated into an Electronic Health Record system for comprehensive patient management. Blood pressure monitors are essential in hospitals to assess cardiovascular health, through continuous monitoring they provide measurement or instant readings of systolic and diastolic blood pressure. Blood pressure monitors identify high blood pressure (hypertension) and helps to diagnose related conditions. Blood pressure monitors could also facilitate early intervention to prevent complications associated with hypertension. Overall the monitor assesses cardiovascular health and alerts healthcare providers.

Through healthcare systems obtaining an alert on a patients heart rate monitor or blood pressure monitor, these devices could communicate to an EHR Electronic Health Record—to show patient statistics of their heart or blood pressure. Connecting these devices to the process of collecting patient data, or filtering data through algorithms outlined in claims, produces an outcome that is specific or novel. Selectively processing this data on specific criteria produces decisions to assist a doctor in a diagnosis or care plan.

Input data in a decision-making algorithm could be obtained or collected through a heart rate monitor or blood pressure monitoring device.

AI Artificial Intelligence systems are often trained on labeled data; an algorithm learns to associate input features to a correct output. The AI chip receives input data from various sources such as patient sensors or user interfaces connected to the patient sensor or monitor (blood pressure monitor connected to an Electronic Health Record). Data collection through a patient monitor assists on an AI algorithm. An AI predictive risk assessment is one component to assess risk of disease progression or complications or readmission of the patient to the hospital.

To continuously monitor a patient involves rings or smartwatches or bracelets to monitor a patients oxygen level or blood pressure, assisting on data collection part of the AI Algorithm. Other ways to monitor a patient providing relevant data is diagnostic equipment including an MRI, CT scanners, X-ray machines to collect detailed patient data.

Laboratory equipment including blood analyzers, microscopes, etc. to collect lab test results.

To connect this collected data to an electronic medical record or to the computing device, perhaps a wireless network including Wi-FI or Bluetooth capabilities to transmit data from sensors to central servers would be optimal. In order for wireless connection to occur, some sort of modem would be necessary. Modems connect to an Internet Service Provider ISP through a cable, fiber, or DSL line—it translates signals from an ISP into data that devices can understand. Network integration occurs through distributing a network interconnection wirelessly to devices such as smartphones, laptops, computers or other smart devices.

Connecting patient monitoring devices to a computing device is central to collect patient data or patient updates such as lab results, blood pressure, oxygenation levels, or other symptoms of underlying diagnoses. Through collecting this sort of data, a decision could be performed on an electronic computer through process outlined in claims section.

An internet connection connected to a modem is a component that assists in the Input or collecting data step, through connecting a computing device to a patient monitoring device, both in two locations of a hospital.

Encryption of data is one component that could assist on Data Privacy, as AI systems process sensitive health information. To encrypt data one must apply an algorithm to securely manage encryption keys, including generating strong encryption keys, storing keys separately from encrypted data, implementing key rotation and revocation policies. Monitoring encryption is also central.

A central server is one component connected to a wireless network, wireless network connected to computing device.

A central server in the context of computing refers to a dedicated machine or system that provides resources, services, or data to other devices (clients) over a network. It acts as a hub for managing and storing information, processing requests, and facilitating communication between users and applications.

Key functions of a central server include:
1. Data Storage: It stores databases, files, and applications that can be accessed by multiple clients.
2. Processing Power: It can perform complex calculations and data processing tasks that may be beyond the capabilities of individual client devices.
3. Resource Management: It manages resources like printers, internet access, and shared applications.
4. Security: It often includes security features to control access and protect data from unauthorized users.
5. Backup and Recovery: It can provide backup services to protect data and ensure recovery in case of failure.

Central servers are connected to a network, using a modem, wherein the network connects to outside computing devices such as a host computer in a hospital.

A central server can interact with AI in several important ways:
1. Data Processing: The server can handle large datasets that AI models require for training and inference. It processes this data efficiently, enabling the AI to learn patterns and make predictions.
2. Model Deployment: Once an AI model is trained, the central server can host it, allowing multiple client devices to access the model for real-time predictions or analysis.
3. Resource Management: AI models often require significant computational power. A central server can provide the necessary hardware (like GPUs or TPUs) to execute complex algorithms efficiently.
4. Scalability: By centralizing AI capabilities, a server can manage multiple client requests simultaneously, allowing it to scale as more users or devices need access to AI features.
5. Updates and Maintenance: Central servers can easily update AI models and algorithms without needing to change software on individual client devices, ensuring everyone has access to the latest advancements.
6. Security and Privacy: The server can manage data access and ensure compliance with privacy regulations, allowing for secure handling of sensitive data used in AI applications.
7. Collaboration: It facilitates collaboration by enabling different clients to share data and insights, improving the AI's performance through collective learning.

Overall, the central server serves as a backbone that enhances the capabilities of AI applications by providing the necessary infrastructure and resources.

A network connected to a central server is composed of Firewalls: Protect the server from unauthorized access and threats.

Encryption: Secures data transmitted over the network to protect sensitive information.

Authentication: Ensures that only authorized clients can access the server and its resources.

Central server is operable to perform the step of collecting an input of patient data through patient monitoring devices such as blood pressure monitors or other continuously patient monitoring devices. Through an internet connection the central server is connected to an external computing device, where a user consults an AI model. No AI model is specific to the computerized system principles outlined in claims section.

A graphical user interface is central component, to verify who the patient is, as well as communicate patient data to doctor. A graphical user interface, or GUI, is a visual interface allowing users to interact with electronic devices using visual elements such as icons (small images that represent programs, files, or functions), menus (lists options or commands that can be accessed with a click), or overall visual representation of data.

A computing device is connected to a cord connected to an outlet for electricity or power supply. An option is a USB is connected to a modem for internet connection.

A computer screen displays patient data or decisional outcomes on the computer proceeding filtration of patient specific data. Data could be collected on an electronic health record. Process is specific to an updated AI (Artificial Intelligence) model algorithm.

An Artificial Intelligence computer chip is comprised of semiconductor substrate that varies depending on the chip but has Silicon, Silicon Dioxide, Copper, Aluminum or other components. a specialized microprocessor to accelerate artificial intelligence (AI) processes.

A keyboard holds numerous buttons where one can choose letters or numbers of words, sentences, or paragraphs or mathematical equations. Keyboards are sometimes wireless, other times wired to the computer itself, where the wire transmits through electricity what is inputted by the keyboard user. Keyboards can also involve shortcuts or buttons that designate a specific task such as closing a window or tab on a graphical user interface. Often these buttons can be designated to specific objectives through a customization option on graphical user interfaces.

A computing device involves electricity, connected through different cords to an outlet or supply of electricity. Computing devices involve a computer screen where the graphical user interface is shown to user. Computing devices are sometimes wired to a desktop where they require a cord connected to an outlet. Other times computing devices are wireless however still require a cord in order to charge electricity. Through a connection to a network, computing devices can access an internet connection, perhaps through a modem. Through an internet connection the computing device can be connected to a central server where encryption of data could occur. In hospitals encryption of data is important to protect patient privacy or other sensitive data.

A central server is connected to a host computer through an internet connection where access could be obtained to an AI model.

Patient data is collected through a patient monitoring device or doctors input through a keyboard. Doctor can type into the keyboard a query and the AI model is trained to solve that input or query through filtering data parallel to how the AI database trained that model. A heart rate monitor or blood pressure monitor could be connected to the central server through an internet connection using a modem, and connected to the host computer through this internet connection. This input of data causes the AI model to process the data parallel to algorithm process outlined in claims section.

### Sensors→Detect Respiratory Decline→Cross-Check Opioid Dose/Timing→Trigger Alert

Drug-Ventilation Conflict Check—Alerts of new opioid prescription could suppress breathing in borderline patients

Process step by step—
1) Ventilator constantly measures—Breathing rate or how fast the patient is breathing. Oxygen levels or how much oxygen the patient needs. Lung pressure. AI reads this data in real time similar to a smartwatch tracking heart rate.
2) AI checks the patients medications—AI has a list of risky drugs that can affect breathing such as Painkillers (morphine, fentanyl) that can slow breathing. Muscle Relaxants such as vecuronium can weaken breathing muscles. Sedatives such as propofol, midazolam can make the patient breathe less. AI also checks patients medication list from the hospital computer system.
3) AI spots problems and alerts doctors. If patient is on a risky drug and the ventilator shows ‘breathing is too slow’, AI warns doctor how Morphine might be causing slow breathing. If ventilator shows lungs are too stiff, AI warns doctor how fentanyl could be making chest muscles tight. If ventilator shows Oxygen is dropping—AI suggests check if sedation is too deep.
4) AI helps adjust the ventilator as an optional step. In this advanced system, AI could automatically adjust settings like ‘increasing oxygen if a drug is lowering breathing’ or ‘reducing pressure if a muscle relaxant is weakening the lungs. Doctor then has the option to approve changes.

Example—patient is on a ventilator and gets morphine for pain. AI sees breathing rate drop from 12 to 6 breaths/minute. Oxygen levels start falling. AI Alert—Morphine may be slowing breathing—consider reducing dose or giving a reversal drug (naloxone).

This system catches drug problems prior to them getting dangerous. It helps doctors remember all drug risks and helps doctors pick safer medications.

Ventilator sends breathing data—AI connects to hospital EHR—checks patient meds-sends alerts to doctors device.

### 1. Ventilator Monitor

Watches:
Breathing rate
Oxygen levels
Lung pressure

### 2. Drug Checker

Knows risky meds (e.g., morphine→slows breathing).
Gets the patient's med list from EHR.

### 3. Alert Generator

If breathing+drugs look dangerous→pings the doctor.
Example: *“Morphine dose too high—breathing rate dropping!”*

Ventilator detects drop in breathing rate, next the AI checks HER sees patient is on morphine. AI matches pattern to opioid-induced respiratory depression. Alert is sent to doctor to adjust settings on ventilator.

Sensor data is collected elsewhere than the ventilator itself. Sensors such as pulse oximeters, CT Scanners, or blood pressure monitors or more could produce data to assist in ventilation settings as they outline a patients health over time.

A ventilator consists of—
Endotracheal Tube (ETT) or Tracheostomy Tube—Direct airway connection.
Non-Invasive Masks—For CPAP/BIPAP modes (e.g., nasal/oral masks)
Flow Sensor—Measures air volume entering/leaving the patient.
Pressure Transducer—Monitors airway pressure (peak, plateau, PEEP).
Oxygen Sensor—Verifies FiO₂ accuracy.
Capnography (Optional)—Measures exhaled CO₂ (end-tidal CO₂).

Spirometry—Tracks lung compliance/resistance. Graphical User Interface (GUI) & Control System
Touchscreen/Display Panel—Primary interface for settings/alarms.
Physical Knobs/Buttons—Backup controls for critical adjustments (e.g., O₂ %, tidal volume).
Alarm Systems—Visual/audible alerts for disconnections, pressure changes, or power failure.
Data Ports/Network—For firmware updates or integration with hospital EMR (electronic medical records).
Water Trap—Collects condensation from humidified air.

Patients present to the emergency department (ED) with a wide range of conditions that may require tracheal intubation or positive-pressure ventilation, including pneumonia, asthma, chronic obstructive pulmonary disease (COPD), cardiogenic pulmonary edema, acute respiratory distress syndrome (ARDS), stroke, trauma, drug overdose, sepsis, shock, and neuromuscular disorders such as myasthenia gravis or Guillain-Barré syndrome.

Once a definitive airway has been secured, ventilatory management ensues. Ventilatory strategies vary according to the clinical scenario, and to provide optimal care, emergency clinicians must understand the fundamental concepts of mechanical ventilation.

The present invention comprises a real-time monitoring and alert system that detects potentially dangerous interactions between pharmaceutical agents and mechanical ventilator settings in critical care environments. At its core, the system integrates three key technological components: (1) a continuous data acquisition module that interfaces with both ventilator systems and medication delivery platforms, (2) a dynamic conflict detection engine containing a specialized knowledge base of drug-ventilator interactions, and (3) a multi-tiered alerting mechanism with clinical decision support capabilities.

The system establishes secure electronic communication with mechanical ventilators through standard hospital interfaces (HL7, FHIR, or manufacturer-specific APIs), continuously monitoring operational parameters including but not limited to ventilation mode (e.g., assist-control, pressure support), respiratory rate, tidal volume, PEEP, FiO₂, and inspiratory/expiratory ratios. Simultaneously, the system receives real-time medication data from electronic health records (EHRs), infusion pumps, and pharmacy systems, tracking all drugs administered to ventilated patients with particular attention to respiratory depressants (e.g., opioids, benzodiazepines), neuromuscular blockers, and stimulants (e.g., doxapram).

The conflict detection engine employs a hybrid approach combining rule-based algorithms with machine learning models trained on clinical outcome data. The rule-based component contains hundreds of validated drug-ventilator interaction pairs derived from pharmacological principles and clinical guidelines (e.g., “fentanyl infusion>50 mcg/hr contraindicated with pressure support ventilation due to apnea risk”). The machine learning component analyzes historical patient data to identify subtle or context-dependent interactions that may not be captured by static rules. Risk stratification occurs through probabilistic scoring, with interactions classified as high (immediate intervention required), medium (clinical evaluation recommended), or low (informational only).

When the system detects a potentially hazardous interaction, it initiates a cascading alert protocol. Primary alerts appear visually on both the ventilator interface and central nursing station dashboards, using color-coded indicators (red for high risk) and specific warning messages (e.g., “WARNING: Rocuronium infusion active in spontaneous breathing mode—risk of ventilator failure”). Secondary alerts are transmitted to clinician mobile devices via secure hospital networks. The system provides context-specific clinical decision support, suggesting appropriate ventilator setting adjustments (e.g., “Recommended action: Switch to volume-controlled ventilation”) or alternative medication regimens.

Advanced embodiments incorporate closed-loop control functionality where the system can automatically implement ventilator setting changes for critical interactions after a configurable delay period (e.g., automatically switching from pressure support to assist-control mode when high-dose opioids are initiated). The system maintains comprehensive audit logs documenting all detected interactions, clinician responses, and system-initiated changes for quality assurance and regulatory compliance.

The user interface presents information through an intuitive dashboard that visualizes risk levels across multiple patients in an ICU, with drill-down capability to view detailed interaction analyses. A unique feature is the drug-ventilator compatibility matrix display, which shows real-time safety status of all active medications against current ventilator settings. The system also includes simulation capabilities, allowing clinicians to preview how proposed medication changes might affect ventilation requirements before administration.

Implementation occurs through a modular software architecture that integrates with existing hospital infrastructure without requiring ventilator hardware modifications. The system undergoes continuous self-validation by comparing its interaction alerts against actual clinical outcomes (e.g., desaturation events, blood gas abnormalities) to refine its detection algorithms. For particularly complex cases, the system can initiate telemedicine consultations with critical care pharmacists or pulmonologists.

This invention represents a significant advancement over existing medication safety systems by addressing the previously unmonitored intersection of pharmaceutical therapy and mechanical ventilation. By preventing dangerous mismatches between drug effects and ventilator support, the system reduces iatrogenic complications, decreases ICU length of stay, and improves overall patient safety in mechanically ventilated populations. The technology is particularly valuable in environments where sedation and ventilation are frequently adjusted, such as ICUs, operating rooms, and long-term acute care facilities.

1) A hospital AI-guided closed-loop ventilator device comprising:
(a) a mechanical ventilator comprising:
(i) an endotracheal tube or tracheostomy tube for direct airway connection;
(ii) a flow sensor that measures air volume entering and leaving a patient;
(iii) a pressure transducer that monitors airway pressure;
(iv) an oxygen sensor that verifies FiO₂ accuracy;
(v) a graphical user interface for displaying settings and patient data; and
(vi) a data port for network integration;
(b) at least one patient sensor comprising a pulse oximeter or capnography device configured to transmit physiological data;
(c) an artificial intelligence processing unit configured to:
(i) continuously receive respiratory data from the mechanical ventilator and physiological data from the at least one patient sensor;
(ii) identify a respiratory condition comprising asthma or a drug-induced respiratory compromise;
(iii) select a condition-specific ventilation protocol corresponding to the identified condition state;
(iv) detect a high-risk interaction between an administered medication and the patient's ventilation status through AI processing; and
(v) output an automatic ventilation mode switch to mitigate the detected interaction, wherein the switch comprises automatically adjusting at least one of an inspiratory flow rate or a plateau pressure according to a ventilation protocol specific to the identified respiratory condition.

2-7) (canceled)
